(19) United States
(12) Patent Application Publication
(10) Pub. No.: US 2003/0050566 A1
(43) Pub. Date: Mar. 13, 2003

(54) Arrhythmia notification

(75) Inventors: Michael R. Ujhelyi, Maple Grove, MN (US); Rahul Mehra, Stillwater, MN (US); Michael F. Hess, Minneapolis, MN (US); Nirav Vijay Sheth, Coon Rapids, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 09/948,368

(22) Filed: Sep. 7, 2001

(51) Int. Cl. A61B 5/0464

(52) U.S. Cl. 600/515

(57) ABSTRACT

Notifying a patient or another person of an arrhythmia episode facilitates management of atrial fibrillation (AF) and other arrhythmias, including atrial flutter, atrial tachycardia, and supra ventricular tachycardia, thus enabling the patient to take corrective action even in the absence of symptoms or latent cardiac problems. For example, the patient may be prompted to take a medication, to initiate electrical therapy in the form of pacing or defibrillation, or to seek medical attention. Notification may be issued either by an implantable medical device or by an external device in communication with the implantable medical device. Various types of notifications may be issued under a variety of conditions, some of which may be associated with the duration of an episode.

### FIELD

The invention relates to implantable medical devices. More particularly, the invention relates to implantable medical devices for treatment of cardiac arrhythmias.

### BACKGROUND

The heart functions by generating electrical signals to initiate physical contractions of various portions of the heart in a specific and timed sequence. These electrical signals are generated by the sinus node in the upper right atrial wall near the base of the heart and are conducted through the upper heart chambers, i.e., the right and left atria, and cause them to contract in a synchronous manner.

The contractions force the blood contained in the atria into the right and left ventricles, or lower heart chambers. An electrical depolarization wave then travels through and around the ventricles, causing the ventricles to contract and force the blood throughout the vascular system. The contraction of the right and left ventricles proceeds in an organized fashion that optimizes emptying of the ventricles.

The synchronous electrical depolarization of the atrial and ventricular chambers can be electrically sensed and displayed, and the electrical waveform is characterized by accepted convention as the “PQRST” complex. The PQRST complex includes a P-wave, corresponding to atrial depolarization, a QRS-complex, corresponding to ventricular depolarization, and a T-wave, which corresponds to ventricular repolarization. The QRS-complex often, but not always, consists of three separate waves, namely, a Q-wave, an R-wave, and an S-wave.

Certain diseases and conduction disturbances can interfere with the natural conduction system of the heart, leading to bradycardia or tachycardia of a heart chamber. That is, various chambers of the heart may be caused to contract too early or too late with respect to the ideal rhythm. These arrhythmias cause a loss of synchronicity between the contractions of the atrial chambers or of the ventricular chambers and, as a result, a loss of cardiac output due to the timing imbalance.

Atrial fibrillation (AF) is a common type of cardiac arrhythmia characterized by an irregular and sometimes rapid heart rhythm. Many AF episodes are asymptomatic. As a result, patients and physicians alike are often unaware of their occurrence or of their burden, i.e., how much time out of a day is spent in a state of arrhythmia. Undiagnosed or under-diagnosed AF episodes can have a number of adverse effects, including chronic AF, congestive heart failure, and thromboembolic events.

Risk increases as the duration of AF episodes increases. For example, AF episodes that last longer than 48 hours present an increased thromboembolic risk. Such long-lasting AF episodes are also more difficult to correct. More generally, long AF durations, especially at faster heart rates, may increase the risk of heart failure and decrease the quality of life of the patient. Moreover, poorly diagnosed AF can lead to inappropriate or ineffective drug therapy. For these reasons, undiagnosed and untreated AF events may place patients at undue risk.

Some conventional devices are able to detect and store AF episodes using mode switching records or direct electrogram storage. A health care provider can analyze this stored information to determine a course of treatment during an office visit. A patient, however, might only make an office visit once every few months in the absence of symptoms. Because many AF episodes do not present symptoms, they may go unnoticed for several months.

### SUMMARY

The invention is directed to devices and processes for notifying a patient or another person of an arrhythmia episode to facilitate management of arrhythmia by allowing the patient to take corrective action even in the absence of symptoms including latent cardiac problems. This notification may address the problem of patient and physician unawareness of arrhythmia episodes, including, but not limited to, atrial fibrillation (AF), atrial flutter, atrial tachycardia, and supra ventricular tachycardia. Effectiveness of AF management may be improved as a result.

For example, notification that an AF or other arrhythmia episode is occurring may prompt a patient to take a medication, to initiate electrical therapy in the form of pacing or defibrillation, or to seek medical immediate attention. By identifying and treating AF episodes when they occur, the likelihood of recurring AF episodes may be reduced. Further, the risk of thromboembolism may also be decreased, reducing or eliminating the need for anticoagulant therapy.

According to various embodiments of the invention, notification may be issued either by an implantable medical device or by a device external to the implantable medical device. Various types of notifications may be issued under a variety of conditions, some of which may be associated with the duration of an episode. These notifications may include, for example, an audible alert, a mechanical (vibration) alert, a displayed notification, or any combination of these types of notifications.

One embodiment of the invention is directed to an implantable medical device that includes a detection module to detect an arrhythmia state in a patient. A duration counter measures a duration of the arrhythmia state. A notification module issues a notification when the duration exceeds a threshold.

In another embodiment, an implantable medical device system includes a detection module to detect an arrhythmia state in a patient. A duration counter measures a duration of the arrhythmia state. An alert module notifies the patient when the duration exceeds a threshold. A status indicator module external to the patient and in communication with the alert module displays an indicator of a detected arrhythmia state.

Still another embodiment of the invention relates to a method for notifying a patient of a detected arrhythmia state. When the arrhythmia state is detected, its duration is measured. When this duration exceeds a threshold, an alert is issued. This method may be implemented as a set of instructions executed by a microprocessor that controls an implantable medical device.

The above summary of the invention is not intended to describe every embodiment of the invention. The details of one or more embodiments of the invention are set forth in the accompanying drawings and the description below.

Other features, objects, and advantages of the invention will be apparent from the description and drawings, and from the claims.

### BRIEF DESCRIPTION OF DRAWINGS

FIG. 1 is a diagram illustrating an implantable medical device system.

FIG. 2 is a block diagram depicting an implantable medical device system incorporating an internal arrhythmia notification module, according to one embodiment of the invention.

FIG. 3 is a functional schematic diagram of an implantable PCD.

FIG. 4 is a flow diagram illustrating an example mode of operation of the PCD of FIG. 3.

### DETAILED DESCRIPTION

In this detailed description, reference is made to the accompanying drawings, which form a part hereof, and in which are shown by way of illustration specific embodiments in which the invention may be practiced. It is to be understood that other embodiments may be utilized and structural or logical changes may be made without departing from the scope of the invention. The following detailed description, therefore, is not to be taken in a limiting sense, and the scope of the invention is defined by the appended claims.

The invention is generally directed to techniques for notifying a patient or another person of an arrhythmia episode, e.g., an episode of atrial fibrillation (AF), atrial flutter, atrial tachycardia, or supra ventricular tachycardia, to facilitate management of arrhythmia by allowing the patient to take corrective action even in the absence of symptoms. This notification may address the problem of patient and physician unawareness of arrhythmia episodes. Effectiveness of arrhythmia management may be improved as a result. For example, notification that an AF or other arrhythmia episode is occurring may prompt a patient to take a medication, to initiate electrical therapy in the form of pacing or defibrillation, or seek medical attention. By identifying and treating AF episodes when they occur, the likelihood of recurring AF episodes may be reduced. Further, the risk of thromboembolism may also be decreased, reducing or eliminating the need for anticoagulant therapy.

Notification may be issued either by an implantable medical device or by a device external to the implantable medical device. Various types of notifications may be issued under a variety of conditions, some of which may be associated with the duration of an episode.

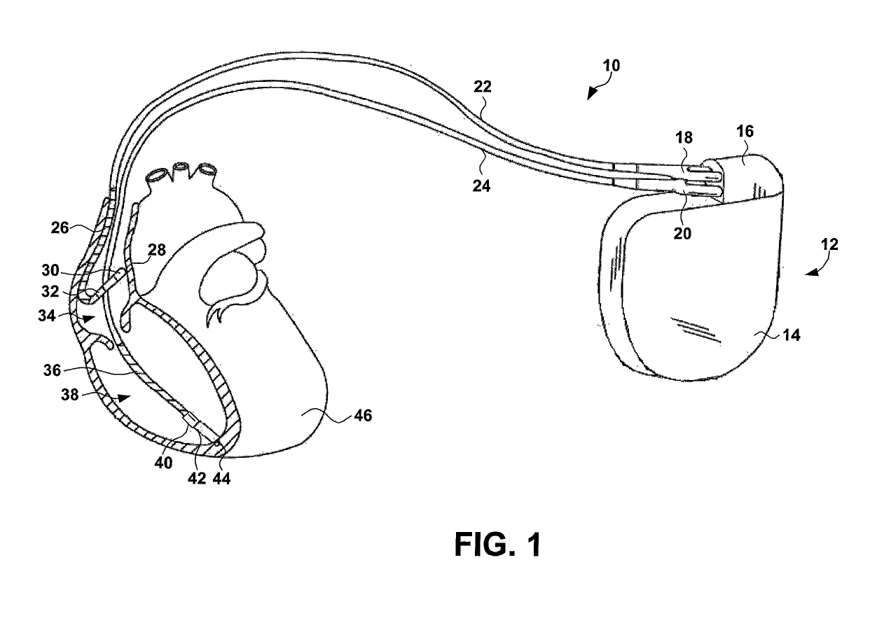

FIG. 1 illustrates an exemplary implantable medical device (IMD) system 10 in which the present invention may be practiced. IMD system 10 is shown in association with a human heart 46. As shown in FIG. 1, IMD system 10 includes a pacemaker/cardioverter/defibrillator (PCD) 12 having a housing 14 and a connector block 16. IMD system 10 may be implemented using any of a number of medical devices or alternative device configurations, including, but not limited to, PCD 12 or an implantable cardiac defibrillator (ICD). Other techniques or therapies responsive to electrocardiogram (EGM) signals, such as therapies that administer drugs in response to atrial arrhythmia, also may implement various embodiments of the invention.

IMD system 10 comprises a ventricular lead, which includes an elongated insulated lead body 24, carrying three concentric coiled conductors separated from one another by tubular insulative sheaths. The distal end of the ventricular lead is deployed in right ventricle 38. Located adjacent the distal end of the ventricular lead are a ring electrode 40, an extendable helix electrode 44, mounted retractably within an insulative electrode head 42, and an elongated (approximately 5 cm) defibrillation coil electrode 36. Defibrillation electrode 36 may be fabricated from many materials, such as platinum or platinum alloy. Each of the electrodes is coupled to one of the coiled conductors within lead body 24.

Electrodes 40 and 44 are employed for cardiac pacing and for sensing ventricular depolarizations. Accordingly, electrodes 40 and 44 serve as sensors for a ventricular electrocardiogram (V-EGM). At the proximal end of the ventricular lead is a bifurcated connector 20 that carries three electrical connectors, each coupled to one of the coiled conductors.

The atrial/superior vena cava (SVC) lead includes an elongated insulated lead body 22, carrying three concentric coiled conductors, separated from one another by tubular insulative sheaths, corresponding to the structure of the ventricular lead. The distal end of the atrial/SVC lead is deployed in right atrium 34. Located adjacent the distal end of the atrial/SVC lead are a ring electrode 32 and an extendable helix electrode 28, mounted retractably within an insulative electrode head 30. Each of the electrodes is coupled to one of the coiled conductors within lead body 22. Electrodes 28 and 32 are employed for atrial pacing and for sensing atrial depolarizations. Accordingly, electrodes 28 and 32 serve as sensors for an atrial electrocardiogram (AEGM).

An elongated coil electrode 26 is provided proximal to electrode 32 and coupled to the third conductor within lead body 22. Electrode 26 is preferably at least 10 cm long and is configured to extend from the SVC toward the tricuspid valve. At the proximal end of the lead is a bifurcated connector 18 that carries three electrical connectors, each coupled to one of the coiled conductors.

Implantable PCD 12 is shown in combination with the leads, with lead connector assemblies 18 and 20 inserted into connector block 16. Optionally, insulation of the outward facing portion of housing 14 of PCD 12 may be provided using a plastic coating, such as parylene or silicone rubber, as is employed in some unipolar cardiac pacemakers. However, the outward facing portion may instead be left uninsulated, or some other division between insulated and uninsulated portions may be employed. The uninsulated portion of the housing 14 optionally serves as a subcutaneous defibrillation electrode, used to defibrillate either the atria or ventricles. Accordingly, the techniques of the present invention may be implemented in medical device systems either with or without leads.

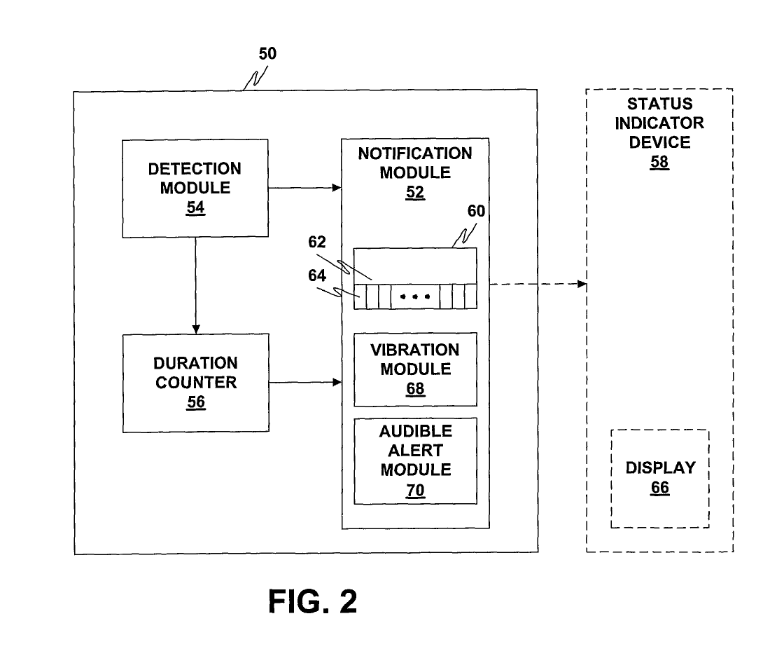

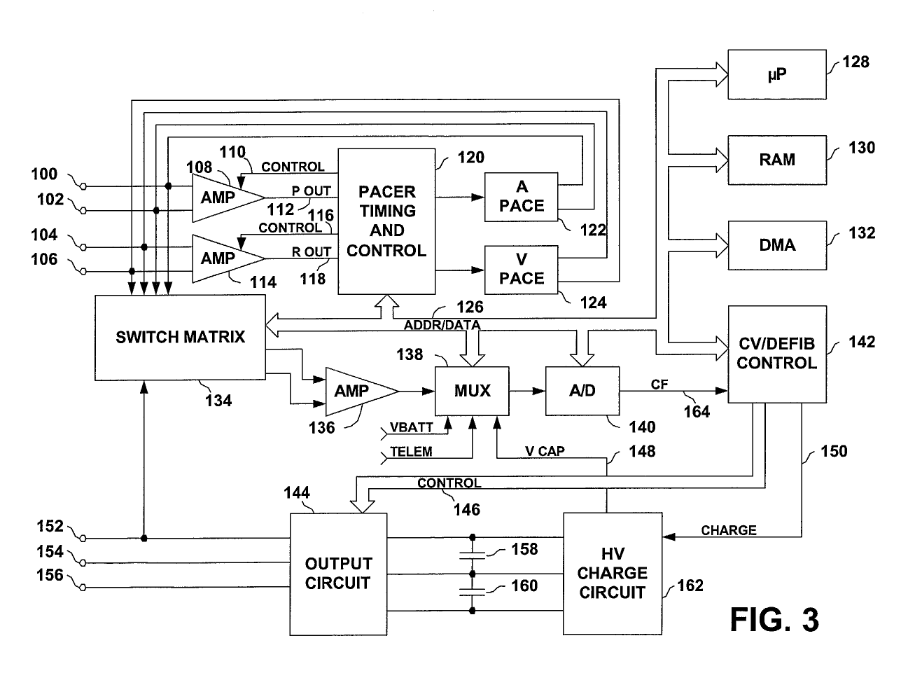

In one embodiment of the invention, PCD 12 detects an arrhythmia state and measures its duration. If the duration exceeds a threshold, PCD 12 issues an alert, either to the patient or to some other person, such as a family member or health care provider. As shown in FIGS. 2 and 3, the alert may be delivered either by PCD 12 itself or by some device external to PCD 12, such as a status indicator device or an alphanumeric pager. The detected arrhythmia state may relate to atrial fibrillation (AF) or to some other type of arrhythmia, such as, for example, atrial flutter, atrial tachycardia, or supra ventricular tachycardia. As described more fully below, the arrhythmia state may relate to any of a number of characteristics of the detected arrhythmia, including, but not limited to, the onset time of the arrhythmia, its duration, and the frequency with which arrhythmia episodes occur. In addition, PCD 12 may also indicate what type of therapy, if any, is administered in response to the detected arrhythmia state, as well as whether the therapy is effective in remedying an episode.

FIG. 2 is a block diagram depicting an implantable medical device system 50 incorporating an internal arrhythmia notification module 52, according to one embodiment of the invention. Notification module 52 may be implemented as microprocessor 128 of FIG. 3, optionally in conjunction with a speaker or vibration module (not shown in FIG. 3). A detection module 54 detects atrial fibrillation (AF) using any of a variety of conventional techniques. Accordingly, the arrhythmia notification features of the present invention may be incorporated into any implantable arrhythmia monitoring device that is capable of detecting AF, either with or without leads.

When detection module 54 detects the onset of an AF episode, a duration counter 56 begins timing the duration of the episode. If this duration exceeds a threshold, notification module 52 issues a notification to the patient or to another person. The threshold may be adjustable, for example, within a range of one minute to 48 hours, and may be set by a physician. By issuing a notification only when the duration exceeds the threshold, notification module 52 avoids being triggered in response to transitory arrhythmias or other arrhythmias that are of insufficient duration to be of clinical interest.

The notification can take a variety of forms. For instance, notification module 52 may include a vibration module 68 to generate a mechanical or vibratory alert. In addition, notification module 52 may select one of a set of programmed tones and tone sequences to alert the patient according to the particular arrhythmia state using an audible alert module 70. Alternatively, a single tone may be sounded to alert the patient to activate an external status indicator device 58 to query notification module 52 for the type of arrhythmia state detected. Status indicator device 58 may require patient activation to query notification module 52 for the arrhythmia state, or may query notification module 52 automatically without having been activated by the patient.

Status indicator device 58 may advise the patient of the arrhythmia state using audible alerts, vibration alerts, light emitting diodes (LEDs), or an alphanumeric display 66, or may use a combination of two or more of these alert mechanisms. Status indicator device 58 may be implemented, for example, as a wristwatch-style device that is worn on the wrist of the patient. In addition, status indicator device 58 may be implemented as an alphanumeric pager carried by a physician, family member, or other person. While FIG. 2 depicts one status indicator device 58, notification module 52 may simultaneously activate multiple status indicator devices 58. For example, notification module 52 may simultaneously activate a device worn by the patient and a pager carried by a physician.

Notification module 52 can be programmed to issue the notification under selected circumstances. For example, notification module 52 may be programmed to issue an alert whenever an arrhythmia episode occurs, regardless of whether a corresponding ventricular response also occurs. As an alternative, notification module 52 may only issue an alert when the episode is accompanied by a ventricular response. This option may be advisable for patients having chronic AF. As another alternative, notification module 52 may be programmed to issue an alert when an AF episode is detected and to issue a subsequent alert when a ventricular response occurs within a short time window after the onset of the AF episode. Notification module 52 may also be programmed to issue another alert when an AF episode ends, either in response to therapy or spontaneously.

Other aspects of the operation of notification module 52 may be programmably modified. For example, the AF episode duration required to trigger an alert may be programmed. Notification module 52 can also be programmed to issue a notification not only regarding the onset of an AF episode, but also regarding any therapy administered. For instance, notification module 52 may be programmed to issue alerts regarding low voltage therapies and to advise the patient as to whether such therapies are effective. Notification module 52 can also be programmed to black out or temporarily suspend an alert that would normally issue. This feature may be useful, for example, if the patient does not want to be alerted while sleeping.

As a particular example of a programmable option, notification module 52 can be programmed to issue an alert upon onset of an AF episode, i.e., to issue an alert before any pacing therapies are administered. Alternatively, notification module 52 may be programmed to only issue an alert after automatic pacing therapies fail.

Notification module 52 may also be programmed to issue an alert if certain burden conditions are satisfied. That is, notification module 52 may issue an alert only if a threshold portion of a given 24-hour period is spent in an AF episode. This threshold may be expressed as a percentage, e.g., 1-100%, of the most recent 24-hour period.

As another alternative, notification module 52 may be programmed to issue tiered alerts, which includes an arrhythmia state relating to a combination of onset, post pacing, and burden criteria. For example, notification module 52 might issue an alert only if an AF episode lasts more than one hour, and then only if at least 25% of the most recent 24-hour period was spend in an AF episode.

Notification module 52 may also incorporate a repeat alert function. In this mode, an arrhythmia state that triggers an additional alert may be defined as the persistence of an AF episode beyond a threshold duration. For example, notification module 52 may be programmed to alert the patient upon onset of an AF episode and to issue another alert every 60 minutes as long as the AF episode persists.

Notification module 52 may also be programmed to suppress alerts under certain circumstances. For example, the programmer may define certain times of day when no alerts are to be issued, or times when audible alerts are to be avoided such as at night or in a public setting. As another example, the programmer may limit the number of alerts that may be issued during a given day. Notification module 52 suppresses any other alerts that would normally issue after this limit is reached. In addition, the programmer may enable or disable a stop alert function that allows the patient to stop an alert.

In addition to these criteria, ventricular response alert criteria may also be defined. For example, as mentioned above, ventricular response alerts may be either enabled or disabled. If enabled, ventricular response alerts may be set to issue only if certain rate criteria or duration criteria are satisfied. In addition, ventricular response alerts may be set to issue under defined tiered rate criteria, i.e., only if a combination of rate criteria and duration criteria are met. The duration criteria may be either independent or dependent of the rate criteria.

For example, the minimum duration required to trigger an alert may be short if the rate is fast, but long if the rate is slow. As with AF alerts, ventricular response alerts may also incorporate burden criteria, repeat alert criteria, or both. In addition, notification module 52 may be programmed to issue another alert when the ventricular response returns to a normal range. Notification module 52 may also be programmed to suppress ventricular response alerts at certain times of day, or in response to patient deactivation of the alert.

The above-described criteria for triggering a notification may be selected by programming a trigger module 60 incorporated in notification module 52. Trigger module 60 may incorporate a control register 62 that uses control bits 64 to specify which trigger criteria are used to trigger a notification. For example, some control bits may be used to specify the burden criteria that must be met to trigger a notification, while other control bits may specify the criteria for activating a repeat alert. Still other control bits may be used to define times of day when notifications are suppressed. Control register 62 may be implemented, for example, using a portion of a random access memory (RAM) device 130 of FIG. 3.

FIG. 3 is a functional schematic diagram of an implantable PCD in which the present invention may be practiced. FIG. 3 should be construed as an illustrative example of one type of device in which the invention may be embodied. The invention is not limited to the particular type of device shown in FIG. 3, but may be practiced in a wide variety of device implementations, such as a pacemaker or an ICD. In addition, the invention is not limited to the implementation shown in FIG. 3. For example, the invention may be practiced in a system that includes more or fewer features than are depicted in FIG. 3.

The device illustrated in FIG. 3 is provided with an electrode system including electrodes. For clarity of analysis, the pacing/sensing electrodes 100, 102, 104, and 106 are shown as logically separate from pacing/defibrillation electrodes 152, 154, and 156. Further, as described above in connection with FIGS. 1 and 2, pacing/sensing electrodes 100, 102, 104, and 106 may be implemented in conjunction with leads, or may be incorporated in a leadless system, e.g. in the device housing.

Electrodes 152, 154, and 156 may correspond respectively to an atrial defibrillation electrode, a ventricular defibrillation electrode, and the uninsulated portion of the housing of the implantable PCD. Electrodes 152, 154, and 156 are coupled to a high voltage output circuit 144. High voltage output circuit 144 includes high voltage switches controlled by cardioversion/defibrillation (CV/defib) control logic 142 via a control bus 146. The switches within output circuit 144 control which electrodes are employed and which are coupled to the positive and negative terminals of a capacitor bank including capacitors 158 and 160 during delivery of defibrillation pulses.

Electrodes 104 and 106 may be located proximate a ventricle and are coupled to an R-wave sense amplifier 114. Operation of amplifier 114 is controlled by pacing circuitry 120 via control lines 116. Amplifier 114 may perform other functions in addition to amplification, such as filtering signals sensed by electrodes 104 and 106. Amplifier 114 may also include a comparator that compares the input signal to a preselected ventricular sense threshold. Amplifier 114 outputs a signal on an R-out line 118 whenever the signal sensed between electrodes 104 and 106 exceeds the ventricular sense threshold.

Electrodes 100 and 102 may be located on or in an atrium and are coupled to a P-wave sense amplifier 108. Operation of amplifier 108 is controlled by pacing circuitry 120 via control lines 110. Amplifier 108 may perform other functions in addition to amplification, such as filtering signals sensed by electrodes 100 and 102. Amplifier 108 may include a comparator that compares the input signal to a preselected atrial sense threshold, which is usually different from the ventricular sense threshold. Amplifier 108 outputs a signal on a P-out line 112 whenever the signal sensed between electrodes 100 and 102 exceeds the atrial sense threshold.

A switch matrix 134 selectively couples the available electrodes to a wide band (2.5-150 Hz) amplifier 136 for use in signal analysis. Signal analysis may be performed using analog circuitry, digital circuitry, or a combination of both.

A microprocessor 128 controls the selection of electrodes via a data/address bus 126. The selection of electrodes may be varied as desired. Amplifier 136 provides signals from the selected electrodes to a multiplexer 138, which provides the signals to an analog-to-digital (A/D) converter 140 for conversion to multi-bit digital signals and to a random access memory (RAM) 130 under control of a direct memory access (DMA) circuit 132 for storage.

The PCD illustrated in FIG. 3 also contains circuitry for providing cardiac pacing, cardioversion, and defibrillation therapies. For example, pacer timing/control circuitry 120 may include programmable digital counters that control the basic time intervals associated with DDD, VVI, DVI, VDD, AAI, DDI, and other modes of single and dual chamber pacing. Pacer timing/control circuitry 120 may also control escape intervals associated with anti-tachyarrhythmia pacing in both the atrium and the ventricle, employing any of a number of anti-tachyarrhythmia pacing therapies.

Intervals defined by pacing circuitry 120 include, but are not limited to, atrial and ventricular pacing escape intervals, refractory periods during which sensed P-waves and R-waves are ineffective to restart timing of the escape intervals, and pulse widths of the pacing pulses. Microprocessor 128 determines the durations of these intervals based on stored data in RAM 130 and communicates these durations to pacing circuitry 120 via address/data bus 126. Microprocessor 128 also determines the amplitude of pacing pulses and communicates this information to pacing circuitry 120.

During pacing, pacing timing/control circuitry 120 resets its escape interval counters upon sensing P-waves and R-waves as indicated by signals on lines 112 and 118. The escape interval counters are reset in accordance with the selected mode of pacing on time-out trigger generation of pacing pulses by pacer output circuits. These pacer output circuits include an atrial pacer output circuit 122 coupled to electrodes 100 and 102, and a ventricular pacer output circuit 124 coupled to electrodes 104 and 106. Pacing timing/control circuitry 120 also resets the escape interval counters when the pacer output circuits generate pacing pulses, thereby controlling the basic timing of cardiac pacing functions, including anti-tachyarrhythmia pacing. Microprocessor 128 determines the durations of the intervals defined by the escape interval timers and communicates these durations using data/address bus 126. The value of the count present in the escape interval counters when reset by sensed R-waves and P-waves may be used to measure the durations of R-R intervals, P-P intervals, P-R intervals, and R-P intervals. These measurements are stored in RAM 130 and used to detect tachyarrhythmias.

Microprocessor 128 typically operates as an interrupt-driven device under control of a program stored in an associated read only memory (ROM, not shown) and is responsive to interrupts from pacer timing/control circuitry 120 corresponding to the occurrence of sensed P-waves and R-waves and to the generation of cardiac pacing pulses. Data/address bus 126 provides these interrupts. In response to these interrupts, microprocessor 128 performs any necessary mathematical calculations, and pacer timing/control circuitry 120 may update the values or intervals that it controls.

When an anti-tachyarrhythmia pacing regimen is indicated based on a detected atrial or ventricular tachyarrhythmia, appropriate timing intervals are loaded from microprocessor 128 into pacer timing/control circuitry 120. In the event that generation of a cardioversion or defibrillation pulse is required, microprocessor 128 employs an escape interval counter to control timing of such cardioversion and defibrillation pulses, as well as associated refractory periods.

In response to the detection of atrial, ventricular fibrillation or tachyarrhythmia requiring a cardioversion pulse, microprocessor 128 activates cardioversion/defibrillation control circuitry 142, which uses high voltage charging control lines 150 to cause a charging circuit 162 to initiate charging of high voltage capacitors 158 and 160. A VCAP line 148 monitors the voltage on high voltage capacitors 158 and 160 and communicates this information through multiplexer 138. When this voltage reaches a predetermined value set by microprocessor 128, A/D converter 140 generates a control signal on Cap Full (CF) line 164 to terminate charging. Thereafter, pacer timing/control circuitry 120 controls timing of the delivery of the defibrillation or cardioversion pulse. Following delivery of the fibrillation or tachyarrhythmia therapy, microprocessor 128 returns the device to cardiac pacing and waits for a subsequent interrupt due to pacing or the occurrence of a sensed atrial or ventricular depolarization.

An output circuit 144 delivers the cardioversion or defibrillation pulses as directed by control circuitry 142 via control bus 146. Output circuit 144 determines whether a monophasic or biphasic pulse is delivered, the polarity of the electrodes, and which electrodes are involved in delivery of the pulse. Output circuit 144 may include high voltage switches that control whether electrodes are coupled together during delivery of the pulse. Alternatively, electrodes intended to be coupled together during the pulse may simply be permanently coupled to one another, either inside or outside the device housing. Similarly, polarity may be preset in some implantable defibrillators.

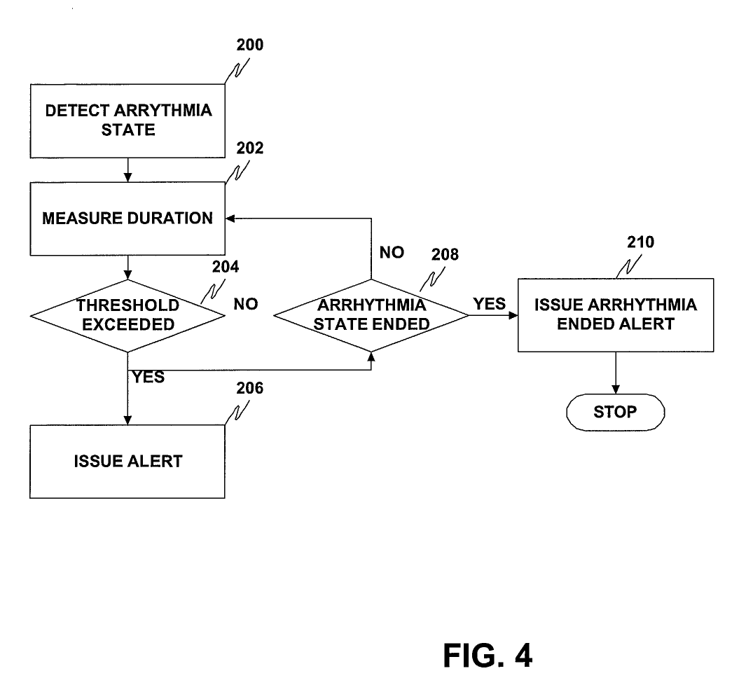

FIG. 4 is a flow diagram illustrating an example mode of operation of the PCD of FIG. 3 to notify a patient or other person of an AF or other arrhythmia episode. This mode of operation may be controlled by a set of instructions executable by microprocessor 128. These instructions may be encoded and stored in a processor-readable medium, including, but not limited to, RAM 130 of FIG. 3, ROM, EEPROM, or flash memory.

As shown in FIG. 4, the PCD detects an arrhythmia state, such as onset of an AF episode or other arrhythmia (200). A duration counter, which may be incorporated as part of pacer timing/control circuitry 120 of FIG. 3, then begins measuring the duration of the arrhythmia state (202). If the duration exceeds a programmable threshold (204), the PCD issues an alert (206). For example, microprocessor 128 of FIG. 3 may cause a speaker to sound an alarm, or may activate a vibration module. Additionally, microprocessor 128 may transmit a signal to an external device to cause the device to display an advisory message. If the duration has not yet exceeded the threshold (204), the duration counter continues measuring the duration (202), unless the arrhythmia state ends (208). When the arrhythmia state ends, the PCD may issue another alert (210).

As discussed above in connection with FIG. 2, the arrhythmia state may be defined in terms of an atrial fibrillation, either in conjunction with or independently of a ventricular response. In addition, the arrhythmia state may also be defined by a threshold duration criterion that must be satisfied, either by an AF episode or by a ventricular response, before an alert issues. The arrhythmia state may also be defined by a threshold burden criterion that must be met, as described above in connection with FIG. 2.

Accordingly, the invention facilitates arrhythmia management by notifying a patient or another person of an arrhythmia episode, thereby allowing the patient to take corrective action even in the absence of symptoms. Identifying and treating AF episodes when they occur may reduce the likelihood of recurring AF episode.

Various embodiments of the invention have been described. These embodiments are illustrative of the practice of the invention. It should be noted, however, that modifications may be made without departing from the scope and spirit of the invention. For example, while several embodiments are described above with respect to AF, the principles of the invention can also be applied to notification of other types of arrhythmias, including, but not limited to, atrial flutter, atrial tachycardia, and supra ventricular tachycardia. These and other embodiments are within the scope of the following claims.

1. An implantable medical device comprising:
a detection module to detect an arrhythmia state in a patient;
a duration counter to measure a duration of the arrhythmia state; and
a notification module to issue a notification when the duration exceeds a threshold.

2. The implantable medical device of claim 1, wherein the notification is issued to the patient.

3. The implantable medical device of claim 1, wherein the notification module comprises at least one of an audible alert module to generate an audible alert and a vibration alert module to generate a vibration alert.

4. The implantable medical device of claim 3, wherein the audible alert is selected as a function of a detected arrhythmia state.

5. The implantable medical device of claim 4, wherein the notification module comprises a trigger module to issue the notification in response to a trigger criterion selected from the group consisting of an arrhythmia onset status, a duration alert, a post automatic therapy failure status, a burden status, a tiered alert status, a repeat alert status, an episode termination alert, and a patient activated stop alert.

6. The implantable medical device of claim 1, wherein the notification module is in data communication with a status indicator module located outside the patient.

7. The implantable medical device of claim 6, wherein the status indicator module comprises a display to indicate the detected arrhythmia state.

8. The implantable medical device of claim 7, wherein the notification module comprises a trigger module to issue the notification in response to a trigger criterion selected from the group consisting of an arrhythmia onset status, a duration alert, a post automatic therapy failure status, a burden status, a tiered alert status, a repeat alert status, an episode termination alert, and a patient activated stop alert.

9. The implantable medical device of claim 7, wherein the indicator comprises an alphanumeric indication of the detected arrhythmia state.

10. The implantable medical device of claim 1, wherein the notification module is further configured to indicate a therapy delivered in response to the detected arrhythmia state.

11. The implantable medical device of claim 10, wherein the notification module is further configured to indicate efficacy of the therapy.

12. The implantable medical device of claim 1, wherein the arrhythmia state relates to atrial fibrillation.

13. An implantable medical device system comprising:
a detection module to detect an arrhythmia state in a patient;
a duration counter to measure a duration of the arrhythmia state;
an alert module to notify the patient when the duration exceeds a threshold; and
a status indicator module to display an indicator of a detected arrhythmia state, the status indicator module external to the patient and in communication with the alert module.

14. The implantable medical device system of claim 13, wherein the notification module comprises at least one of an audible alert module to generate an audible alert and a vibration alert module to generate a vibration alert.

15. The implantable medical device system of claim 14, wherein the audible alert is selected as a function of the detected arrhythmia state.

16. The implantable medical device system of claim 13, wherein the alert module comprises a trigger module to issue the notification in response to a trigger criterion selected from the group consisting of an arrhythmia onset status, a duration alert, a post automatic therapy failure status, a burden status, a tiered alert status, a repeat alert status, an episode termination alert, and a patient activated stop alert.

17. The implantable medical device system of claim 13, wherein the indicator comprises an alphanumeric indication of the detected arrhythmia state.

18. The implantable medical device system of claim 13, wherein the status indicator module is configured to query the detection module for the detected arrhythmia state in response to activation by the patient.

19. The implantable medical device system of claim 13, wherein the status indicator module is configured to query the detection module for the detected arrhythmia state automatically in response to a notification issued by the alert module.

20. The implantable medical device system of claim 13, wherein the alert module is further configured to indicate a therapy delivered in response to the detected arrhythmia state.

21. The implantable medical device system of claim 20, wherein the alert module is further configured to indicate efficacy of the therapy.

22. The implantable medical device system of claim 13, wherein the arrhythmia state relates to atrial fibrillation.

23. A method for notifying a patient of a detected arrhythmia state, the method comprising: detecting the arrhythmia state;
measuring a duration of the arrhythmia state; and
issuing an alert when the duration exceeds a threshold.

24. The method of claim 23, further comprising generating at least one of an audible alert and a vibration alert in response to detecting the arrhythmia.

25. The method of claim 24, further comprising selecting the audible alert as a function of a detected arrhythmia state.

26. The method of claim 25, further comprising issuing an alert in response to a trigger criterion selected from the group consisting of an arrhythmia onset status, a duration alert, a post automatic therapy failure status, a burden status, a tiered alert status, a repeat alert status, an episode termination alert, and a patient activated stop alert.

27. The method of claim 23, further comprising issuing a notification to a status indicator module located outside the patient.

28. The method of claim 27, further comprising causing the status indicator module to display an indicator of a detected arrhythmia state.

29. The method of claim 28, further comprising issuing an alert in response to a trigger criterion selected from the group consisting of an arrhythmia onset status, a duration alert, a post automatic therapy failure status, a burden status, a tiered alert status, a repeat alert status, an episode termination alert, and a patient activated stop alert.

30. The method of claim 28, wherein the indicator comprises an alphanumeric indication of the detected arrhythmia state.

31. The method of claim 23, further comprising indicating a therapy delivered in response to the detected arrhythmia state.

32. The method of claim 31, further comprising indicating efficacy of the therapy.

33. The method of claim 31, wherein the arrhythmia state relates to atrial fibrillation.

34. A processor-readable medium containing processor-executable instructions for:
detecting an arrhythmia state;
measuring a duration of the arrhythmia state; and
issuing an alert when the duration exceeds a threshold.

35. The processor-readable medium of claim 34, containing further processor-executable instructions for generating at least one of an audible alert and a vibration alert in response to detecting the arrhythmia.

36. The processor-readable medium of claim 35, containing further processor-executable instructions for selecting the audible alert as a function of a detected arrhythmia state.

37. The processor-readable medium of claim 36, containing further processor-executable instructions for issuing an alert in response to a trigger criterion selected from the group consisting of an arrhythmia onset status, a duration alert, a post automatic therapy failure status, a burden status, a tiered alert status, a repeat alert status, an episode termination alert, and a patient activated stop alert.

38. The processor-readable medium of claim 34, containing further processor-executable instructions for issuing a notification to a status indicator module located outside the patient.

39. The processor-readable medium of claim 38, containing further processor-executable instructions for causing the status indicator module to display an indicator of a detected arrhythmia state.

40. The processor-readable medium of claim 39, containing further processor-executable instructions for issuing an alert in response to a trigger criterion selected from the group consisting of an arrhythmia onset status, a duration alert, a post automatic therapy failure status, a burden status, a tiered alert status, a repeat alert status, an episode termination alert, and a patient activated stop alert.

41. The processor-readable medium of claim 39, wherein the indicator comprises an alphanumeric indication of the detected arrhythmia state.

42. The processor-readable medium of claim 34, containing further processor-executable instructions for indicating a therapy delivered in response to the detected arrhythmia state.

43. The processor-readable medium of claim 42, containing further processor-executable instructions for indicating efficacy of the therapy.
